(19) United States
(12) Patent Application Publication
(10) Pub. No.: US 2025/0311485 A1
(43) Pub. Date: Oct. 2, 2025

(54) POLARIZATION-INTENSITY COUPLED LIGHT EMITTING DEVICE

(71) Applicant: Yuan LU, Nancy (FR)

(72) Inventor: Yuan LU, Nancy (FR)

(21) Appl. No.: 19/091,546

(22) Filed: Mar. 26, 2025

Related U.S. Application Data
(60) Provisional application No. 63/570,297, filed on Mar. 27, 2024.

(51) Int. Cl. H10H 20/812; H10H 20/814; H10H 20/831; H10H 20/856; H10N 99/00

(52) U.S. Cl. CPC H10H 20/812; H10H 20/8142; H10H 20/831; H10H 20/856; H10N 99/05

(57) ABSTRACT

Disclosed is a polarization-intensity coupled light emitting device. In the light emitting device, a semiconductor structure is configured to generate light in response to carrier injection; a spin injector is configured to inject carriers into the semiconductor structure, wherein the light generated by the semiconductor structure has a circular polarization state determined by the magnetization state of the spin injector; a magnetization controller is configured to change the magnetization state of the spin injector; and a chiral metasurface is configured to make differential response to left-handed circularly polarized light component and right-handed circularly polarized light component of the light generated by the semiconductor structure. When the magnetization direction of spin injector is switched, both intensity and circular polarization of the light from the light emitting device can be modulated simultaneously.

### CROSS-REFERENCE TO RELATED APPLICATIONS

This application claims the benefit of U.S. Provisional Application No. 63/570,297, filed on Mar. 27, 2024, the disclosure of which is incorporated herein by reference in its entirety and for all purposes.

### TECHNICAL FIELD

The disclosure herein relates to a spin-based polarization-intensity coupled light emitting device.

### BACKGROUND

An explosive growth of Information and Communication Technology (ICT) applications, involving artificial intelligence, Big Data, Internet of Things (IoT) and 5G, requires a high-transmission bandwidth with improved energy efficiency provided by optical communication systems.

Datacenters alone are predicted to require 8% of globally generated electrical power by 2030. A critical challenge for today's optical communications is how to continue increasing the modulation bandwidth while keeping the overall energy consumption manageable.

Remarkably, this bottleneck can be overcome by using the conversion between carrier and photon spin in semiconductor lasers. It is reported that an optical injection of spin-polarized carriers in lasers can enable an ultrafast polarization modulation of the emitted light (>200 GHz), much faster than the intensity-modulation in the conventional lasers (20-30 GHz), while simultaneously supporting an ultralow power consumption. This discovery shows the superior advantages for optical communication with the polarization than with the intensity, offering a paradigm shift in spintronics to support novel room-temperature (RT) applications beyond magnetoresistance.

However, actual single mode optic fiber popularly used in Datacenter cannot well conserve the circular polarization during the light propagation. The fiber defect, bending, twisting, and environmental factors can seriously modify the polarization states of light. Therefore, it is necessary to convert the polarization modulation to intensity modulation for transferring the encoded light in the single mode fiber. One standard method is to use a combination of 1/4 waveplate and polarizer before coupling the light into the optical fiber. However, due to the large volume and expensive cost of optic components, this method is not practical for real application in the Datacenter.

### SUMMARY

According to an aspect of the disclosure, a light emitting device is provided. The light emitting device including: a semiconductor structure is configured to generate light in response to carrier injection; a spin injector is configured to inject carriers into the semiconductor structure, wherein the light generated by the semiconductor structure has a circular polarization state determined by the magnetization state of the spin injector; a magnetization controller is configured to change the magnetization state of the spin injector; and a chiral metasurface is configured to make differential response to left-handed circularly polarized light component and right-handed circularly polarized light component of the light generated by the semiconductor structure.

### BRIEF DESCRIPTION OF FIGURES

By more detailed description of the exemplary embodiments of the present disclosure in combination with the accompanying drawings, the above and other purposes, features and advantages of the present disclosure will become more apparent. In the exemplary embodiments of the present disclosure, the same reference numeral generally represents the same component.

FIG. 1 is a schematic view of the light emitting device according to the disclosure.

FIG. 2 is a schematic view showing relative positional relationships of the components of the light emitting device according to an embodiment of the disclosure.

FIG. 3 is a schematic view showing relative positional relationships of the components of the light emitting device according to a variant embodiment of the disclosure.

FIG. 4 shows the selection rule of optical transition in GaAs based semiconductor quantum wells or quantum dots.

FIG. 5 illustrates the magnetization switching in the injector Hall-bar structure by spin Hall effect (SHE).

FIG. 6 is a cross-sectional view of the light emitting device according to an embodiment of the disclosure.

FIG. 7 is an exemplified top view of the light emitting device according to the embodiment of the disclosure.

FIG. 8 is another exemplified top view of the light emitting device according to the embodiment of the disclosure.

FIG. 9 is a cross-sectional view of the light emitting device according to a variant embodiment of the disclosure.

FIG. 10 is a cross-sectional view of the light emitting device (spin-VCSEL) according to another variant embodiment of the disclosure.

FIG. 11 is an exemplified top view of the spin-VCSEL according to the variant embodiment of the disclosure.

FIG. 12 is another exemplified top view of the spin-VCSEL according to the variant embodiment of the disclosure.

FIGS. 13A and 13B show the intensity of light with right-handed circular polarization (σ+) and light with left-handed circular polarization (σ−) as a function of injection current J in various situations without metasurface based chiral reflector. FIG. 13A shows the situation when injector M is switched in up direction and FIG. 13B shows the situation when injector M is switched in down direction.

FIGS. 14A and 14B show the intensity of light with right-handed circular polarization (σ+) and light with left-handed circular polarization (σ−) as a function of injection current J in various situations with metasurface based chiral reflector. FIG. 14A shows the situation when injector M is switched in up direction and FIG. 14B shows the situation when injector M is switched in down direction.

### DETAILED DESCRIPTION

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

The semiconductor spintronics technology is used to achieve the light emission with desired circular polarization. By depositing a ferromagnetic layer as a spin injection layer on the top of the quantum wells or quantum dots based LED structure, spin-polarized electrons can be injected into the semiconductor quantum wells or semiconductor quantum dots. The spin-polarized electrons will undergo quantum transition to recombine with holes according to the law of conservation of angular momentum, and thus circularly polarized photons will be emitted. Each of the semiconductor quantum wells or semiconductor quantum dots is capable of emitting photons with circular polarization direction determined by the spin direction of the injected spin-polarized carriers.

In this disclosure, a novel spin-based polarization-intensity coupled light emitting device is provided with a chiral metasurface or chiral material layer as an effective polarization filter. With the chiral metasurface or chiral material layer introduced, the polarization modulation can be easily converted to intensity modulation without using any optical components.

The chiral metasurface layer or chiral material layer can be directly integrated into spin light emitting diode (spin-LED) or spin vertical cavity surface emitting laser (spin-VCSEL). Therefore, the volume and cost can be greatly saved.

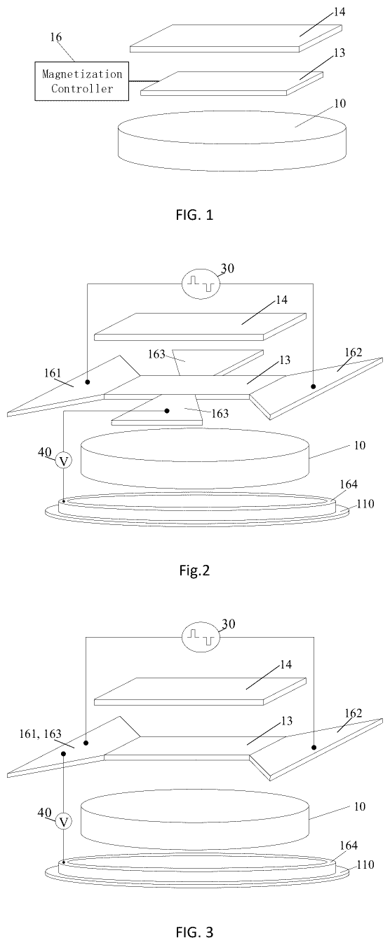

FIG. 1 is a schematic view of the light emitting device according to the disclosure.

As shown in FIG. 1, the light emitting device according to the disclosure is a spin-based light emitting device, including a semiconductor structure 10 and a spin injector 13.

The spin injector 13 is configured to inject carriers into the semiconductor structure. The spin polarization state of the carriers injected from the spin injector 13 is determined by the magnetization state of the spin injector 13.

Here, the carriers can be either electrons or holes. Generally, the electrons are used as the carriers because the spin lifetime of electrons is much longer than that of holes.

The semiconductor structure 10 is configured to generate light in response to carriers injection from the spin injector 13.

In some embodiments, the semiconductor structure 10 may be sandwiched between a substrate 110 (Not shown in FIG. 1 but shown in FIGS. 2-3 and 6-10) and the spin injector 13. When a bias voltage is applied between the spin injector 13 and the substrate 110, carriers will be injected from the spin injector 13 to the semiconductor structure 10.

The light generated by the semiconductor structure 10 has a circular polarization state determined by the magnetization state of the spin injector 13.

In some embodiments, the semiconductor structure 10 includes a gain medium of quantum dots (QD) or quantum wells (QW). The gain medium (quantum dots or quantum wells) are configured to generate light with circular polarization state determined by the spin direction of the injected spin-polarized carriers, in response to the spin-polarized carriers injected into the semiconductor structure 10 and recombined in the gain medium of quantum dots or quantum wells.

The light emitting device may further include a magnetization controller 16 configured to control the magnetization state (especially, the magnetization direction) of the spin injector 13.

It shall be understood that, the spin direction of the spin-polarized carriers injected from the spin injector 13 into the semiconductor structure 10 is determined by the magnetization direction of the spin injector 13.

By changing the magnetization direction of the spin injector 13 through the magnetization controller 16, the spin polarization direction of the carriers injected from the spin injector 13 into the semiconductor structure 10 will change accordingly. And thus, the circular polarization state of the light generated will change accordingly.

In some embodiments, the spin injector 13 is in a form of a bar-shaped channel.

Accordingly, the magnetization controller 16 may include two electrodes respectively connected to two opposite ends of the bar-shaped channel to apply a current pulse into the bar-shaped channel, so as to switch the magnetization direction of the spin injector 13. With the two electrodes, alternating directional pulse current may be applied into the bar-shaped channel to alternatively reverse the magnetization direction of the spin injector 13.

As shown in FIG. 1, the light emitting device according to the disclosure further includes a chiral metasurface 14. The chiral metasurface 14 is configured to make differential response to left-handed circularly polarized light component and right-handed circularly polarized light component of the light generated by the semiconductor structure 10.

In some embodiments, the chiral metasurface 14 is arranged above the spin injector 13.

In some embodiments, the chiral metasurface 14 is arranged on the emission side of the light emitting device, and is configured to allow the light generated by the semiconductor structure 10 to transmit through.

The chiral metasurface 14 is composed of chiral-shaped nanostructures.

In recent years, extensive research has been conducted on chiral metasurface and chiral-shaped nanostructures, with various implementation schemes and effectiveness analyses being proposed. All these specific implementation approaches are applicable in the embodiments disclosed herein, therefore no limitations are imposed on the concrete realization methods of chiral metasurface and chiral-shaped nanostructures, nor will they be reiterated here.

The chiral metasurface 14 is designed to have a strong circular dichroism (CD) in transmission.

When the light emitting device is a spin-based LED, the chiral metasurface 14 is configured to exhibit a higher transmittance for either one of the two polarized light components (i.e., the left-handed circularly polarized light component and the right-handed circularly polarized light component) compared to the other one of the two polarized light components. The transmittance of the left-handed circularly polarized light component versus the right-handed circularly polarized light component is determined by the specific design of the chiral-shaped nanostructure in the chiral metasurface 14.

For the sake of clarity, either left-handed circularly polarized light component or the right-handed circularly polarized light component is referred to as a “first polarized light component”, and the other of left-handed circularly polarized light component or right-handed circularly polarized light component is referred to as a “second polarized light component”.

Therefore, when the light emitting device is a spin-based LED, the chiral metasurface 14 is configured to exhibit a higher transmittance for a first polarized light component compared to a second polarized light component. As mentioned above, the first polarized light component is either the left-handed or right-handed circularly polarized light, while the second polarized light component is the other opposite-handed circularly polarized light.

As an example of the chiral metasurface 13, a 2D arrayed structure with a C4 fourfold rotational symmetry is fabricated by e-beam lithography.

The chiral metasurface 13 can effectively filter either left-handed or right-handed circularly polarized light passing through it.

For example, by using spin-orbit torque (SOT) as will be described below, the circular polarization Pc of the light emitted by the light emitting device can be modulated between +50% and −50% without the metasurface (no light intensity change between the two states).

By introducing the chiral metasurface 13, for example, if the transmission rate of the right-handed circularly polarized light component σ+ is 0.8 and the transmission rate of the left-handed circularly polarized light component σ− is 0.2 (corresponding to a CD of 0.6), the final Pc of the light emitted out of the light emitting device can be modulated between +84.6% and +14.3% with an intensity ratio of 1.857 between the two magnetic states of the spin injector 13.

Therefore, a conversion of polarization modulation to intensity modulation is realized.

FIG. 2 is a schematic view showing relative positional relationships of the components of the light emitting device according to an embodiment of the disclosure.

As shown in FIG. 2, a semiconductor structure 10 is formed above a substrate 110, and a spin injector 13 is formed above the semiconductor structure 10. In other words, the semiconductor structure 10 is sandwiched between the substrate and the spin injector 13.

In some embodiments, the substrate 110 is a semiconductor substrate.

In some embodiments, the semiconductor structure 10 is a III-V semiconductor-based structure.

In some embodiments, there might be some other layers sandwiched between the substrate 110 and the semiconductor structure 10. Or, in other embodiments, the semiconductor structure 10 might be formed directly on the substrate 110.

In some embodiments, there might be some other layers sandwiched between the semiconductor structure 10 and the spin injector 13. Or, in other embodiments, the spin injector 13 might be formed directly on the semiconductor structure 10.

In some embodiments, the spin injector 13 is a metallic spin injector, and may have a Hall-bar structure. the magnetization state of the spin injector 13 can be switched by spin Hall effect (SHE).

In the example of FIG. 2, the spin injector 13 is depicted as in a form of a bar-shaped channel. The magnetization controller 16 includes a first electrode 161 and a second electrode 162. In some embodiments, the magnetization controller 16 may also include the current pulse generator 30. In some embodiments, the current pulse generator 30 is an supplier external to the magnetization controller 16 and applying current pulses to the first electrode 161 and the second electrode 162.

The first electrode 161 and the second electrode 162 may be formed above the semiconductor structure 10 and are respectively connected to two opposite ends of the bar-shaped channel (spin injector 13). Furthermore, the first electrode 161 and the second electrode 162 are respectively connected to two output terminals of a current pulse supplier to receive current pulses, which are then applied to the bar-shaped channel (spin injector 13) to electrically control the magnetization direction of the spin injector 13.

As mentioned above, the spin direction of the spin-polarized carriers injected from the spin injector 13 into the semiconductor structure 10 is determined by the magnetization state of the spin injector 13.

To sum up, the magnetization state of the spin injector 13 can be electrically controlled by applying current pulse into the spin injector 13 via the first electrode 161 and the second electrode 162. The spin polarization state (spin directions) of the carriers injected from the spin injector 13 into the semiconductor structure 10 is thus determined by the magnetization state of the spin injector 13. And accordingly, the circular polarization state of the light generated by the light emitting device in semiconductor structure 10 is determined by the spin polarization state of the injected carriers. In other words, the circular polarization state of the light generated by the light emitting device in semiconductor structure 10 can be alternated by changing the direction of the current pulse applied between the first electrode 161 and the second electrode 162 and flowing through the spin injector 13.

The direction of the current pulse applied into the bar-shaped channel (spin injector 13) can be reversed. The spin injector 13 is configured so that its magnetization direction can be switched by applying a current pulse in the opposite direction to the previously applied current pulse in the spin injector.

In this example, as shown in FIG. 2, the first electrode 161 and the second electrode 162 are respectively connected to the two opposite ends in the lengthwise direction of the bar-shaped channel (spin injector 13), so as to introduce the current pulse into the bar-shaped channel to flow through the lengthwise direction, and thus change the magnetization state (magnetization direction) of the spin injector 13.

Further, a third electrode 163 and a fourth electrode 164 are respectively connected to two output terminals of a voltage source supplier 40 to receive voltage signals.

The third electrode 163 may be formed above the semiconductor structure 10, and is connected to the bar-shaped channel (spin injector 13). And the fourth electrode 164 may be formed on the substrate 110. The third electrode 163 and the fourth electrode 164 are thus configured to apply a voltage (bias voltage) between the spin injector 13 and the substrate 110 to inject carriers from the spin injector 13 into the semiconductor structure 10.

In the example of FIG. 2, two of the third electrodes 163 are shown on two opposite sides in widthwise direction of the bar-shaped channel (spin injector 13). It shall be understood that the third electrode 163 can be formed in many other forms, as long as it is electrically connected to the spin injector 13.

In some embodiments, one or both of the first electrode 161 and the second electrode 162 can serve as the third electrode 163 to apply voltage signal to the spin injector 13. In other words, the first electrode 161 and/or the second electrode 162 can be further connected to one output terminal of the voltage source supplier 40 to receive the voltage signals, in addition to receiving the current pulses.

FIG. 3 is a schematic view showing relative positional relationships of the components of the light emitting device according to a variant embodiment of the disclosure. As shown in FIG. 3, the third electrode 163 and the first electrode 161 share the same electrode. The first electrode 161 also serves as the third electrode 163, and is connected to the voltage source supplier 40 to apply a voltage signal to the spin injector 13.

As shown in FIGS. 2 and 3, the chiral metasurface 14 is formed above the spin injector 13.

In some embodiments, there might be some other layers sandwiched between the spin injector 13 and the chiral metasurface 14. Or, in other embodiments, the chiral metasurface 14 might be formed directly on the spin injector 13.

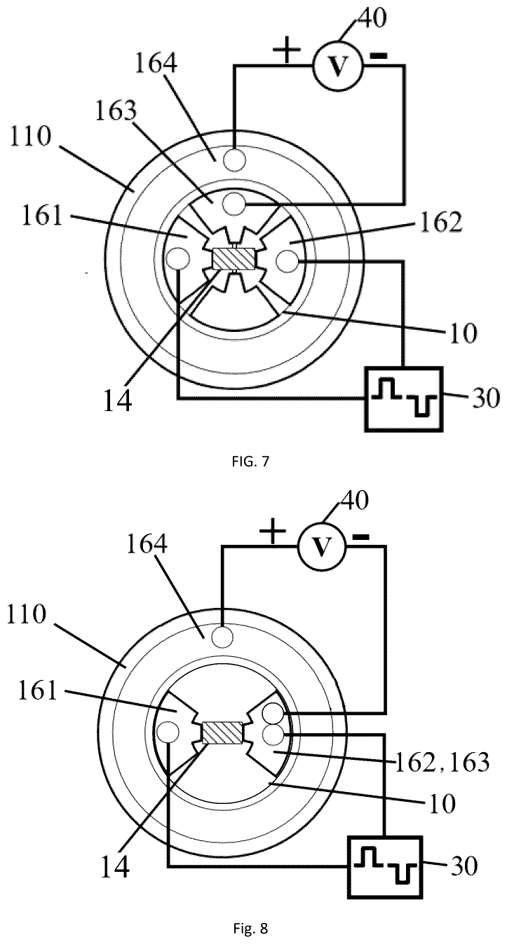

Hereinafter, the selection rule of optical transition and the magnetization switching in the spin injector will be described with reference to FIGS. 5 and 6.

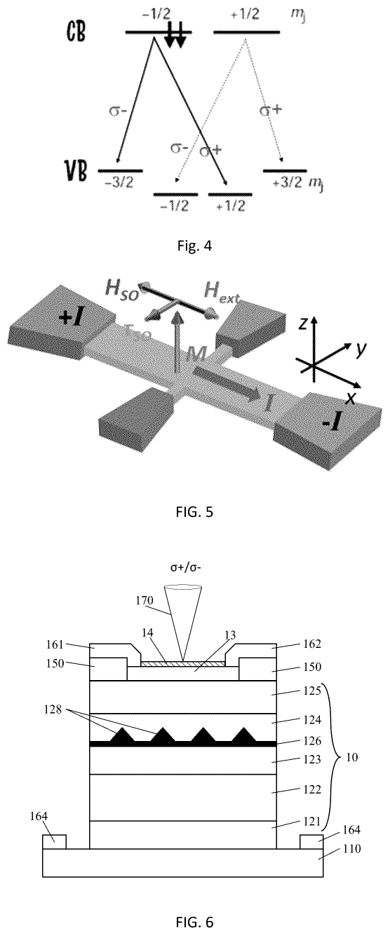

FIG. 4 shows the selection rule of optical transition in GaAs semiconductor quantum wells or quantum dots.

As shown in FIG. 4, when an electron with spin of −1/2 is injected into the conduction band of the semiconductor quantum wells or quantum dots through a ferromagnetic spin injection layer or spin injector (such as CoFeB/MgO layer), according to the conservation law of angular momentum quantum number mⱼ (the change of angular momentum quantum number before and after the transition Δmⱼ=±1), the electron is allowed to transition to the valence band in only two ways.

One way is to recombine with a heavy hole in the heavy hole valence band (mⱼ=−3/2), that is, to transition from mⱼ=−1/2 to mⱼ=−3/2 (Δmⱼ=−1), emitting a left-handed circularly polarized photon, which can be referred to as “σ−”.

The other way is to recombine with a light hole in the light hole valence band (mⱼ=+1/2), that is, to transition from mⱼ=−1/2 to mⱼ=+1/2 (Δmⱼ=+1), emitting a right-handed circularly polarized photon, which can be referred to as “σ+”.

However, in the quantum well structure or the quantum dot structure, the light and heavy hole valence bands are non-degenerated, and the heavy hole transition matrix element (transition probability) is much larger than the light hole transition matrix element (transition probability). Therefore, while an electron with spin of −1/2 is injected, a left-handed circularly polarized photon (σ−) will be obtained with almost 100% probability.

Conversely, while an electron with spin of +1/2 is injected, a right-handed circularly polarized photon (σ+) will be obtained with almost 100% probability.

Therefore, the circular polarization direction of the photon emitted from the quantum wells or quantum dots completely depends on the spin direction of the injected electron. And thus, the circular polarization direction of the light emitted from the semiconductor structure 10 corresponds to the spin polarization direction of the carriers injected into the semiconductor structure 10.

It should be emphasized here that the optical selection rule requires the spin direction to be parallel to the photon emission direction. To obtain a circularly polarized photon without magnetic field, the magnetization direction of the ferromagnetic injection layer (spin injector 13) shall be perpendicular to the sample surface for surface emission geometry.

Based on the above principle, the semiconductor structure 10 is capable of emitting light beam with controllable circular polarization direction by injecting carriers with controlled spin polarization direction into the semiconductor structure 10.

FIG. 5 illustrates the magnetization switching in the spin injector with a Hall-bar structure by SHE.

As shown in FIG. 5, a current I is injected in the designed ferromagnet (FM)/heavy metal (HM) spin-injector channel to generate current-induced spin-orbit torque (SOT) Tso and associated spin-orbit field H_{SO}. With a small in-plane external constant magnetic field Hₑₓₜ, the perpendicular magnetization of injector can be deterministically switched when injecting alternative direction of current in the channel. The magnetization switch can be realized with very short pulse current (6 ps), which allows for high-speed operation. The latest developments in the field of spintronics show the possibility to avoid the application of Hₑₓₜ by using different strategies, such as using spin textured ferromagnetic layer, an in-plane exchange bias or growth on substrates with specific crystalline orientation.

To electrically control the circular polarization direction of the emitted light beam, a current pulse will be applied to the spin injector 13 (through the first electrode 161 and the second electrode 162) to switch the magnetization state of the spin injector 13. Then, the spin injector 13 is negatively biased (through the third electrode 163 and the fourth electrode 164) to enable a light emission.

As the magnetization state changes in response to the applied current pulse, the spin polarization state of the carriers injected from the spin injector 13 into the semiconductor structure 10 also changes accordingly.

According to the optical selection rule, the circular polarization direction (right circular polarization σ+ or left circular polarization σ−) of the light generated from the semiconductor structure 10 will be determined by the spin polarization direction of carriers injected from the spin injector 13. Therefore, by switching the magnetization state of the spin injector 13, the spin polarization direction of the injected carriers can be changed, and the circular polarization direction of the generated light beam will be controlled accordingly.

In embodiments, the magnetization state of the spin injector 13 refers to the magnetization direction of the spin injector 13.

Magnetization direction of the spin injector 13 is flipped by applying the current pulse into the bar-shaped channel of the spin injector 13.

The magnetization state of the spin injector 13 are non-volatile and are capable of being retained after the current pulse are applied.

The parameters of the current pulse, such as amplitude, duration, numbers of sub-pulse and so on, can be configured to make sure that the magnetization direction of the whole spin injector 13 is flipped into one magnetization direction, for example, up-direction (↑).

While a bias voltage is applied between the third electrode 163 and the fourth electrode 164, spin-polarized carriers are injected from the spin injector 13 to the semiconductor quantum wells or semiconductor quantum dots 128 in the semiconductor structure 10.

When the spin injector 13 has an up-direction (↑) magnetization, the majority of carriers injected from the spin injector 13 into the semiconductor structure 10 are polarized to spin of +1/2. Accordingly, the light generated from the semiconductor structure 10 has a positive circular polarization, in which the proportion of the right circularly polarized light (σ+) component is greater than that of the left circularly polarized light component (σ−).

And, when the spin injector 13 has a down-direction (↓) magnetization, the majority of carriers injected from the spin injector 13 into the semiconductor structure 10 are polarized to spin of −1/2. Accordingly, the light generated from the semiconductor structure 10 has a negative circular polarization, in which the proportion of the right circularly polarized light (σ+) component is smaller than that of the left circularly polarized light component (σ−).

In addition, after a light beam is generated from the semiconductor structure 10 with the spin injector 13 in one magnetization direction, the magnetization direction of the spin injector 13 can be flipped to the opposite direction, for example, from up-direction (↑) magnetization to down-direction (↓) magnetization, by applying a further current pulse with a direction opposite to the previous one.

A more detailed structure of the light emitting device will be described below with reference to FIGS. 6-8.

FIG. 6 is a cross-sectional view of the light emitting device according to an embodiment of the disclosure. FIGS. 7 and 8 are exemplified top views of the light emitting device according to the embodiment of the disclosure.

As shown in FIG. 6, the light emitting device includes a semiconductor structure 10, a spin injector 13 and a chiral metasurface 14.

In some embodiments, the semiconductor structure 10 is a GaAs based structure. The semiconductor structure 10 can be a light emitting diode structure with a gain medium layer 128 formed above a wetting layer 126. Quantum wells or quantum dots are formed in the gain medium layer 128. P-doped layers (121, 122) are formed below the gain medium layer 128. And n-doped layer (125) is formed above the gain medium layer 128.

As shown in FIG. 6, a semiconductor structure 10 is formed on a substrate 110. The substrate 110 might be a p-doped GaAs substrate with a (001) crystal plane, i.e., a p-GaAs (001) substrate.

The semiconductor structure 10 includes, from bottom to top, a p-doped GaAs (p-GaAs) layer 121 (for example, 300 nm), a p-doped A_{0.3}Ga_{0.7}As layer 122 (for example, 400 nm), a Be δ-doping layer 123 (for example, 30 nm), a wetting layer 126 of InGaAs, a gain medium layer 128 with quantum wells or quantum dots formed therein, a GaAs layer 124 (for example, 50 nm) and a n-doped GaAs layer 125 (for example, 50 nm).

In some embodiments, the semiconductor structure 10 is cylindrical, and when viewed from top down, it might be disc-shaped.

The spin injector 13 is formed on the top layer of the semiconductor structure 10, i.e., the n-doped GaAs layer 125. And the chiral metasurface 14 is formed on the spin injector 13.

As shown in FIG. 6, the light beam 170 is emitted out of the light emitting device from the chiral metasurface 14. The chiral metasurface 14 is arranged on the emission side of the light emitting device, and is configured to allow the light generated by the semiconductor structure 10 to transmit through.

As described above, the spin injector 13 might be in form of a bar. The sizes (length and width) of the upper surface of the bar-shaped spin injector 13 are smaller than the radius of upper surface of the cylindrical semiconductor structure 10.

FIG. 7 is an exemplified top view of the light emitting device according to the embodiment of the disclosure.

As shown in FIG. 7, a first electrode 161 and a second electrode 162, as well as a third electrode 163 are formed surrounding the spin injector 13 (covered by the chiral metasurface 14 in the top view shown in FIG. 7), and connected with the spin injector 13.

The fourth electrode 164 is formed on the substrate 110. The fourth electrode 164 might be a ring shape surrounding the cylindrical semiconductor structure 10 with an interval.

As described above, the first electrode 161 and the second electrode 162 are respectively connected to two opposite ends of the bar-shaped channel of the spin injector 13 to apply current pulses into bar-shaped channel of the spin injector 13.

The first electrode 161 and the second electrode 162 are further connected to a current pulse generator (or a current pulse source) 30. The current pulse generator 30 provides the current pulses with a direction corresponding to the circular polarization direction of the light beam desired to be emitted. And the current pulse generator 30 is capable of alternatively reversing the direction of the current pulses to change the circular polarization direction of the generated light beam.

The third electrode 163 and the fourth electrode 164 are configured to apply a bias voltage between the spin injector 13 and the substrate 110. A voltage source supplier 40 may be configured to provide the bias voltage (V).

In some embodiments, the first electrode 161 and/or the second electrode 162 may also serve as the third electrode 163 to receive the bias voltage with respect to the fourth electrode 164. In other words, the first electrode 161 and/or the second electrode 162 can be further connected to one output terminal of the voltage source supplier 40 to receive the voltage signals, in addition to receiving the current pulses.

FIG. 8 is another exemplified top view of the light emitting device according to the embodiment of the disclosure.

As shown in FIG. 8, the third electrode 163 and the second electrode 162 share the same electrode. The second electrode 162 further serves as the third electrode 163, is connected to the voltage source supplier 40 to apply voltage signal to the spin injector 13 (covered by the chiral metasurface 14 in the top view shown in FIG. 8).

An insulating material layer 150 is formed between the electrodes 161, 162, 163 and the top layer of the semiconductor structure 10 and surrounding the spin injector 13, insulating the electrodes 161, 162 and 163 from the top layer of the semiconductor structure 10. The insulating material layer 150 might be formed by SiO₂.

In some embodiments, the first electrode 161, the second electrode 162, the third electrode 163 and the fourth electrode 164 are formed from Ti, or Au or combination of Ti and Au such as double-layer film (Ti/Au) or Ti—Au alloy.

By applying the current pulse into the spin injector 13 via the first electrode 161 and the second electrode 162, the magnetization state (magnetization direction) of the spin injector 13 can be changed accordingly.

And then, by applying a bias voltage (voltage) between the spin injector 13 and the substrate 110 via the third electrode 163 and the fourth electrode 164, spin-polarized carriers will be injected from the spin injector 13 into the semiconductor structure 10, especially into the quantum wells or quantum dots (gain medium layer 128).

In response to the spin-polarized carriers, the quantum wells or quantum dots 128 generate light with circular polarization (σ+ or σ−) state corresponding to the spin polarization state of the carriers.

The chiral metasurface 14 is configured to exhibit a higher transmittance for either the left-handed circularly polarized light component or the right-handed circularly polarized light component compared to the other opposite-handed polarized light component.

And thus, the light emitting device will emit a light beam 170 with circular polarization state further modulated by the chiral metasurface 14.

As described above, by using spin-orbit torque (SOT) as will be described below, the circular polarization Pc of the light emitted by the light emitting device can be modulated between +50% and −50% without the metasurface (no intensity change between the two states).

By introducing the chiral metasurface 13, for example, if the transmission rate of the right circularly polarized light component σ+ is 0.8 and the transmission rate of the left circularly polarized light component σ− is 0.2 (corresponding to a CD of 0.6),

Specifically, suppose the total intensity of the light generated by the semiconductor structure 10 is I₀.

When circular polarization Pc of the generated light is +50%, intensity I₁ᵣ of the right-handed circularly polarized light component σ+ is 0.75I₀ and intensity I₁ₗ of the left-handed circularly polarized light component σ− is 0.25I₀.

The total intensity I₁ of the light beam after transmitting through the chiral metasurface 14 is

$${I}_{1} = {I}_{1 r} + {I}_{1 l} = 0.75 {I}_{0} \times 0.8 + 0.25 {I}_{0} \times 0.2 = 0.65 {I}_{0}$$

And, the circular polarization Pc₁ of the light beam after transmitting through the chiral metasurface 14 is

$${Pc}_{1} = \frac{{I}_{1 r} - {I}_{1 l}}{{I}_{1 r} + {I}_{1 l}} = \frac{0.75 {I}_{0} \times 0.8 - 0.25 {I}_{0} \times 0.2}{0.75 {I}_{0} \times 0.8 + 0.25 {I}_{0} \times 0.2} \times 100 \% = + 84.6 \%$$

When circular polarization Pc of the generated light is −50%, intensity I₂ᵣ of the right-handed circularly polarized light component σ+ is 0.25I₀ and intensity I₂ₗ of the left-handed circularly polarized light component σ− is 0.25I₀.

The total intensity I₂ of the light beam after transmitting through the chiral metasurface 14 is

$${I}_{2} = {I}_{2 r} + {I}_{2 l} = 0.25 {I}_{0} \times 0.8 + 0.75 {I}_{0} \times 0.2 = 0.35 {I}_{0}$$

The intensity ratio of I₁ to I₂ is

$$\frac{{I}_{1}}{{I}_{2}} = \frac{0.65 {I}_{0}}{0.35 {I}_{0}} = 1.857$$

And, the circular polarization Pc₂ of the light beam after transmitting through the chiral metasurface 14 is

$${Pc}_{2} = \frac{{I}_{2 r} - {I}_{2 l}}{{I}_{2 r} + {I}_{2 l}} = \frac{0.25 {I}_{0} \times 0.8 - 0.75 {I}_{0} \times 0.2}{0.25 {I}_{0} \times 0.8 + 0.75 {I}_{0} \times 0.2} \times 100 \% = + 14.3 \%$$

The final Pc of the light beam emitted out of the light emitting device can be modulated between +84.6% and +14.3% with an intensity ratio of 1.857 between the two magnetic states of the spin injector 13. A conversion of polarization modulation to intensity modulation is realized.

As mentioned above, the light emitting device as shown in FIG. 6 may be referred to “spin-LED”.

The semiconductor structure 10 may be in a form of multi-layer mesa. FIGS. 6, 7 and 8 show only one light emitting device formed on one multi-layer mesa with one spin injector 13.

In some embodiments, a plurality of light emitting devices can be formed on one multi-layer mesa. The plurality of light emitting devices share the multi-layer semiconductor mesa as the semiconductor structure 10.

In a variant embodiment, a bottom distributed Bragg reflector (DBR) may be formed under the semiconductor structure 10.

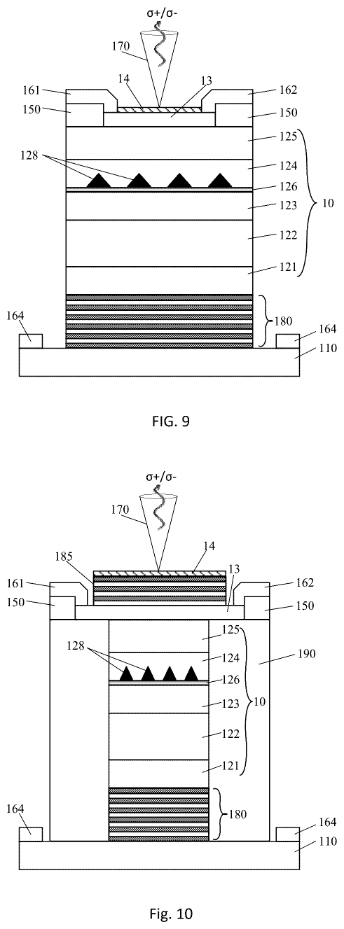

FIG. 9 is a cross-sectional view of the light emitting device according to a variant embodiment of the disclosure. The light emitting device shown in FIG. 9 is a spin-LED with bottom DBR to enhance the light intensity.

As compared with the embodiment as shown in FIG. 6, in the variant embodiment shown in FIG. 9, a bottom distributed Bragg reflector (DBR) 180 is arranged below the semiconductor structure 10. In other words, the semiconductor structure 10 is sandwiched between the spin injector 13 and the bottom distributed Bragg reflector (DBR) 180.

By arranging the bottom distributed Bragg reflector (DBR) 180 below the semiconductor structure 10, the light output efficiency from surface emitting direction can be enhanced.

The Distributed Bragg reflector (DBR) is a periodic structure composed of two materials with different refractive index arranged alternately in the stack form of ABAB. The product of reflection index and the thickness of each layer of material is equal to 1/4 of the central reflection wavelength.

The bottom distributed Bragg reflector (DBR) 180 may be formed as a dielectric Bragg reflector structure by alternative deposition of different materials such as AlGaAs/AlAs.

The light emitting device as shown in FIG. 9 can be referred to as “spin 1/2-VCSEL”. In another variant embodiment, a spin-VCSEL can be formed.

FIG. 10 is a cross-sectional view of the light emitting device according to another variant embodiment of the disclosure. The light emitting device as shown in FIG. 10 can be referred to as “spin-VCSEL”, an optical cavity is formed between a bottom DBR and a top DBR.

The optical cavity provides several unique advantages: (1) lasing threshold reduction, i.e., the lasing threshold J_{T1} is reduced compared to lasing threshold J_{T} (unpolarized threshold) of conventional lasers; and (2) spin amplification, i.e., the circular polarization (Pc) of the light emitted from the light emitting device is greater than the spin polarization (Pn) of the carriers injected from the spin injector 13. Remarkably, for injection between majority spin threshold J_{T2} and minority spin threshold J_{T1}, J_{T1}<J<J_{T2}, even a small spin polarization (Pₙ) will lead to circular polarization rate (Pc) of approximately 100%. Indeed, experiments yield circular polarization rate (Pc) of about 96% for spin polarization (Pn) of about 4%.

As compared with the embodiment as shown in FIG. 9, in the variant embodiment as shown in FIG. 10, a top distributed Bragg reflector (DBR) 185 is arranged above the spin injector 13. In other words, the spin injector 13 is sandwiched between the semiconductor structure 10 and the top distributed Bragg reflector (DBR) 185. The chiral metasurface 14 is formed above the top distributed Bragg reflector (DBR) 185.

By further arranging atop distributed Bragg reflector (DBR) 185 above the spin injector 13, an intracavity resonant surface emitting laser structure is formed between the top distributed Bragg reflector (DBR) 185 and the bottom distributed Bragg reflector (DBR) 180. And, a Vertical-Cavity Surface Emitting Laser (VCSEL) is thus formed. Such a micro-scale resonant cavity can greatly improve the light emission efficiency of the quantum wells or the quantum dots.

The top distributed Bragg reflector (DBR) 185 may be formed as a dielectric Bragg reflector structure by alternative deposition of different materials such as TiO₂/Al₂O₃, CaF₂/ZnS, MgF₂/ZnS.

In order to form a spin-VCSEL structure (FIG. 10), the semiconductor part, which includes the bottom DBR 180 and the semiconductor structure 10 with an embedded gain medium (quantum wells or quantum dots), is firstly formed on the substrate 110. And then, the semiconductor part is surrounded by photoresist 190 (for example, benzocyclobutene (BCB)) with a flat surface. The diameter of the bottom DBR 180 and the semiconductor structure 10 might be about 20 μm.

The spin injector 13 will then be deposited on the top surface of the semiconductor part and the photoresist 190 and followed by lithography process to form the Hall-bar structure. The surface area of the spin injector 13 is large enough to cover all the semiconductor structure 10 to ensure a homogenous current injection into the gain medium region for laser emission.

To minimize the optical absorption risk due to the inserted metallic spin injector 13 in the cavity, the distance between the spin injector 13 and the active region (i.e. the gain medium of quantum dots or quantum wells) is configured so that the spin injector 13 is placed in one node of the stationary electromagnetic field formed by the light reflected between the top and bottom distributed Bragg reflectors.

The top DBR 185 is then deposited on the spin injector 13 to complete the optical cavity.

And then, the chiral metasurface 14 is formed on the top DBR 185.

The chiral metasurface 14 is configured to exhibit a higher reflectivity for a first polarized light component compared to a second polarized light component, wherein the first polarized light component is either the left-handed circularly polarized light component or the right-handed circularly polarized light component, and the second polarized light component is the other opposite-handed circularly polarized light.

The reflectivity of the left-handed circularly polarized light component versus the right-handed circularly polarized light component is determined by the specific design of the chiral-shaped nanostructure in the chiral metasurface 14. In other word, whether the left-handed circularly polarized light component or the right-handed circularly polarized light component has a higher reflectivity is determined by the specific design of the chiral-shaped nanostructure of the chiral metasurface 14.

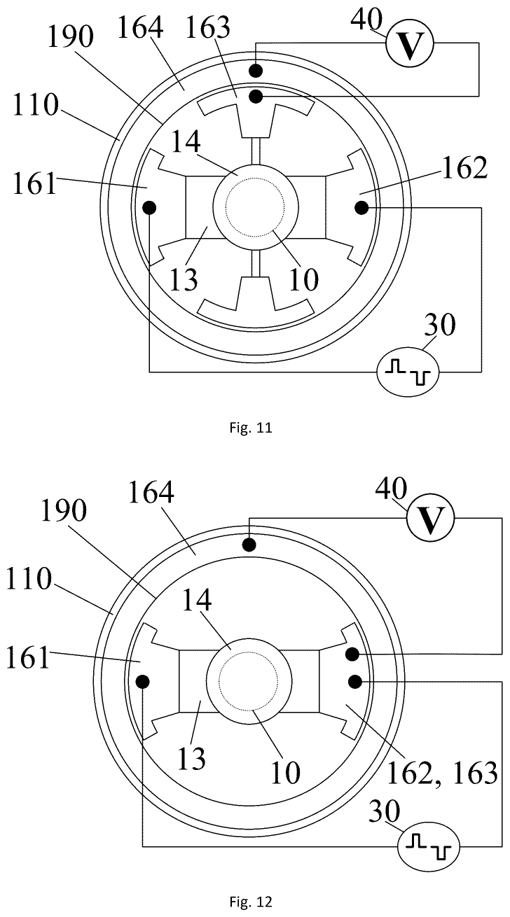

FIG. 11 is an exemplified top view of the spin-VCSEL according to the variant embodiment of the disclosure.

As shown in FIG. 11, the semiconductor structure 10 (covered by the spin injector 13 and the chiral metasurface 14, and drawn with dotted line) is surrounded by the photoresist 190.

The spin injector 13, the first electrode 161, the second electrode 162 and the third electrode 163 are formed on the top surface of the semiconductor structure 10 and the surrounding photoresist 190.

The spin injector 13 is a form of a bar-shaped channel. And, the first electrode 161 and the second electrode 162 are respectively connected to the two opposite ends in the lengthwise direction of the bar-shaped spin injector 13.

The third electrode 163 is also connected to the spin injector 13. And, the fourth electrode 164 is formed on the substrate 10. The fourth electrode 164 might be a ring shape surrounding the cylindrical semiconductor structure 10 (and the bottom DBR below the cylindrical semiconductor structure 10) with an interval.

The first electrode 161 and the second electrode 162 are further connected to a current pulse generator (or a current pulse source) 30 to apply the current pulse into the spin injector 13. The third electrode 163 and the fourth electrode 164 are connected to a voltage source supplier 40 to apply the voltage signal between the substrate 110 and the spin injector 13.

The metasurface 14 covers the top DBR 185 (not shown in FIG. 11), and is large enough to cover all the semiconductor structure 10 to ensure a homogenous current injection into the gain medium region for laser emission.

In the example shown in FIG. 11, the semiconductor structure 10 is formed in a cylindrical shape, the metasurface 14, as well as the top DBR 185, may have a round disc shape with a larger radius than that of the semiconductor structure 10.

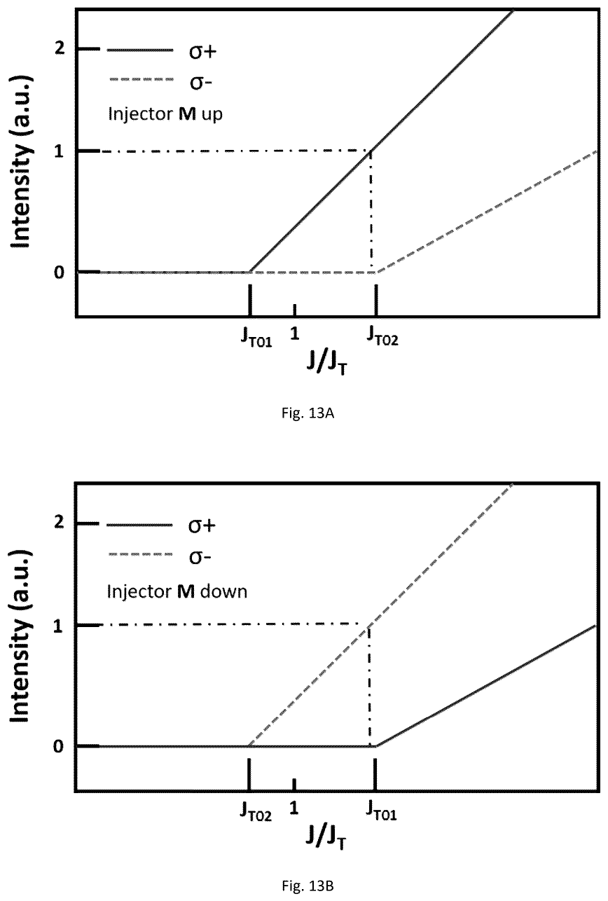

FIG. 12 is another exemplified top view of the spin-VCSEL according to the variant embodiment of the disclosure.

FIG. 12 differs from FIG. 11 in that the third electrode 163 and the second electrode 162 share the same electrode. The second electrode 162 further serves as the third electrode 163, is connected to the voltage source supplier 40 to apply voltage signal to the spin injector 13.

In operating the spin VCSEL shown in FIG. 10 and FIG. 11 or 12, current pulse will be sent into the spin injector via the first electrode 161 and the second electrode 162 to switch the magnetization state of spin injector 13. The spin injector 13 will be negatively biased by the third electrode 163 and the fourth electrode 164 for a continuous emission. Circular polarization state (σ+ and σ−) of the light generated by the semiconductor structure 10 will be modulated according to the pulsed-current direction in the channel of spin injector 13.

The light generated by the semiconductor structure 10 will bounce back and forward between the bottom DBR 180 and the top DBR 185, thereby achieving oscillation amplification.

According to basic laser physics, cavity mirror reflectivity can influence the threshold gain gₜₕ of a VCSEL, which is given by:

$${g}_{th} = \frac{1}{Γ} [ {α}_{i} + \frac{1}{2 {L}_{eff}} ln ( \frac{1}{{R}_{1} {R}_{2}} ) ]$$

Where Γ is the confinement factor, αᵢ is the internal loss, L_{eff} is the effective cavity length, and R₁ and R₂ are the power reflectivities of the top and bottom mirrors, respectively. When the reflectivity of the top mirror (top DBR 185 and the chiral metasurface 14) differs for the two orthogonal circular polarizations (the left-handed circularly polarized light component and the right-handed circularly polarized light component), the threshold gain values for the two polarization modes will be different, resulting in chirality-dependent lasing behavior.

Specially, if the reflectivity for right-handed circular polarization (RCP) is higher than that of left-handed circular polarization (LCP), the threshold gain for RCP will be relatively lower. As a result, the VCSEL will prefer to lase in RCP. And vice versa.

The lasing threshold is defined as the minimum carrier injection current required to initiate laser emission. The carrier injection current is the current of carriers injected upon the application of the voltage between the spin injector 13 and the substrate 110.

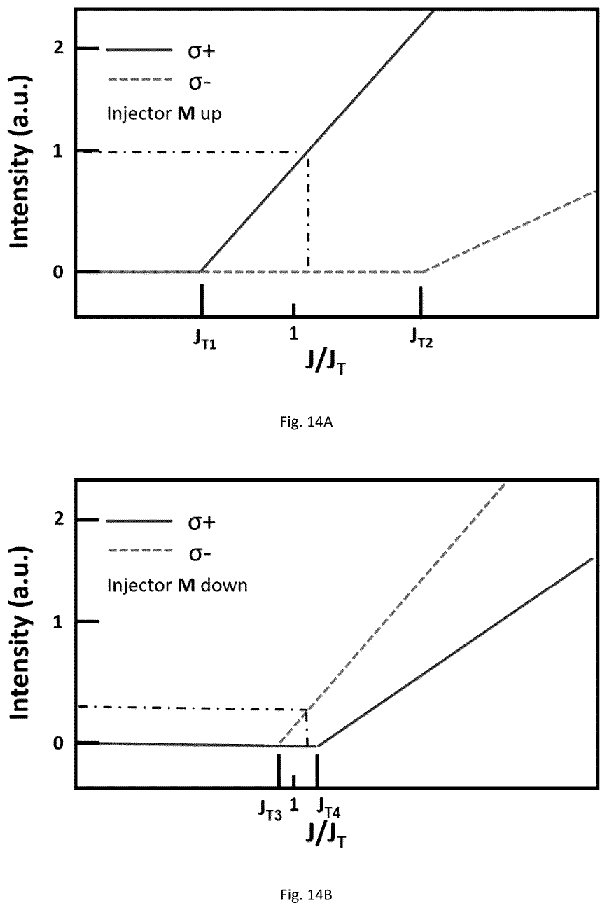

FIG. 13A to 14B show the intensity of light of right-handed circular polarization (σ+) and left-handed circular polarization (σ−) as a function of injection current J in various situations. The injection current J is normalized to the unpolarized threshold J_{T}.

Wherein, FIGS. 13A and 13B show the relationships between the intensity of light and injection current J for normal spin-VCSEL without introducing the chiral metasurface 14, while FIGS. 14A and 14B show the relationship between the intensity of light and injection current J for spin-VCSEL of the disclosure with the chiral metasurface 14 introduced as described above.

FIGS. 13A and 14A show the relationships between the intensity of light and injection current J while the magnetization direction (M) of the spin injector is the upward direction, while FIGS. 13B and 14B show the relationships between the intensity of light and injection current J while the magnetization direction (M) of the spin injector is the downward direction.

As shown in FIGS. 13A and 13B, for normal spin-VCSEL, due to the spin amplification effect, the lasing threshold for right-handed circularly polarized light σ+ (J_{T01}) and the lasing threshold for left-handed circularly polarized light σ− (J_{T02}) are different.

Working in the current region between J_{T01} and J_{T02} allows to obtain a circular polarization (Pc) of nearly 100%. When SOT switching injector M, the Pc can be modulated between +100% to −100%. However, the intensity of light almost keeps constant when magnetization direction (M) of the spin injector 13 is switched up or down, with the injection current J remaining the same.

The situation is changed with the combination of the chiral metasurface 14 which acts as a chiral reflector, as shown in FIGS. 14A and 14B.

Supposing the chiral metasurface 14 has a slight larger reflectivity for right circularly polarized light σ+, it will favorite σ+ laser emission.

When the magnetization direction (M) of the spin injector 13 is in upward state, both the effects of spin amplification and chiral reflection will enlarge the difference between the two thresholds (J_{T1} and J_{T2}). However, when the magnetization direction (M) of the spin injector 13 is in downward state, the both effects will cancel together, resulting in a reduced difference in the two thresholds (J_{T3} and J_{T4}).

As a consequence, if an appropriate injection current J between the two thresholds (J_{T1} and J_{T2}, or J_{T3} and J_{T4}) is applied, not only a large circular polarization modulation, but also a large 200-300% intensity modulation can be obtained by switching the injector magnetization.

As shown in FIG. 14A, by applying an injection current J slightly larger than the unpolarized threshold J_{T}, the light emitted by the light emitting device has a circular polarization of +100% (completely right-handed circularly polarized, σ+) and an intensity of 1 (A.U., Arbitrary Unit). And as shown in FIG. 14B, by applying the same injection current J, the light emitted by the light emitting device has a circular polarization of −100% (completely left-handed circularly polarized, σ−) and an intensity of about 0.32.

To be more general, the light emitting device is a spin-VCSEL with a chiral metasurface reflector (the chiral metasurface 14), and has four lasing thresholds of carrier injection current satisfying the following relationship.

$${J}_{T 1} < {J}_{T 3} < {J}_{T 4} < {J}_{T 2} .$$

Wherein, J_{T1} is a first lasing threshold of carrier injection current for the first polarized light component when the spin injector 13 is in a first magnetization state. And as defined above, the first polarized light component is the polarized light component for which the chiral metasurface 14 is configured to exhibit a higher reflectivity compared to the other polarized light component. For example, the first magnetization state has an upward magnetization direction, while the first polarized light component is right-handed circularly light component σ+.

J_{T2} is a second lasing threshold of carrier injection current for the second polarized light component when the spin injector 13 is the first magnetization state.

When the spin injector 13 is in the first magnetization state, no light is emitted if the carrier injection current is lower than first lasing threshold J_{T1}, light having only the first polarized light component is emitted if the carrier injection current is between the first lasing threshold J_{T1} and the second lasing threshold J_{T2}, and light having both the first polarized light component and the second polarized light component is emitted if the carrier injection current is larger than the second lasing threshold J_{T2}

J_{T3} is a third lasing threshold of carrier injection current for the second polarized light component when the spin injector 13 is in a second magnetization state. For example, the second magnetization state has a downward magnetization direction, while the second polarized light component is left-handed circularly light component σ−.

J_{T4} is a fourth lasing threshold of carrier injection current for the first polarized light component when the spin injector 13 is in the second magnetization state.

When the spin injector 13 is in the first magnetization state, no light is emitted if the carrier injection current is lower than first lasing threshold J_{T1}, light having only the first polarized light component is emitted if the carrier injection current is between the first lasing threshold J_{T1} and the second lasing threshold J_{T2}, and light having both the first polarized light component and the second polarized light component is emitted if the carrier injection current is larger than the second lasing threshold J_{T2}.

Further, compared with the two thresholds J_{T01} and J_{T02} of normal spin-VCSEL without a metasurface, there is a further relationship as follow.

$${J}_{T 1} < {J}_{TS} < {J}_{T 3} < {J}_{T 4} < {J}_{TL} < {J}_{T 2} .$$

Wherein, J_{TS} is the smaller one of J_{T01} and J_{T02}, and J_{TL} is the larger one of J_{T01} and J_{T02}.

The voltage applied between the spin injector 13 and the substrate is configured such that the carrier injection current J is between J_{T3} and J_{T4}:

$${J}_{T 1} < {J}_{T 3} < J < {J}_{T 4} < {J}_{T 2} .$$

Supposing that I₁ is a first intensity of the light emitted from the light emitting device with the first polarized light component when the spin injector 13 is in the first magnetization state, and I2 is a second intensity of the light emitted from the light emitting device with the second polarized light component when the spin injector 13 is in the second magnetization state, there is an inequality relation as follow:

I₁>I₂.

Therefore, an intensity modulation can be realized by a polarization modulation.

That is to say, when a larger intensity (I₁) of light is expected to be emitted by the light emitting device, the spin injector 13 is switched into the first magnetization state. On the other hand, when a smaller intensity light (I₂) is expected to be emitted by the light emitting device, the spin injector 13 is switched into the second magnetization state.

Since the polarization modulation speed (>200 GHz) of spin-based light emitting device is much faster than the intensity-modulation speed (about 20-30 GHz) in the conventional lasers, a faster intensity modulation can be realized by a conversion of polarization modulation to intensity modulation by introducing a chiral metasurface 14 in the spin-VCSEL.

The combination of chiral metasurface 14 on a spin-LED or a spin-VCSEL can generate several important advantages compared to use SOT spin-LED and spin-VCSEL only for polarization modulation.

Since the chiral metasurface 14 can effectively acts as a polarized light filter for spin-LED and polarization dependent reflector for spin-VCSEL, it can efficiently convert polarization modulation into intensity modulation without using any optical components, this will largely save the system volume and reduce the cost of product.

For a standard spin-VCSEL, the CPL could be influenced by the residual cavity parameters rather than the spin injection. The introducing of chiral reflector will force the eigenstates in VCSEL to be helicity eigenstates, which favors the ultrafast polarization modulation dynamics in spin-VCSEL.

For spin-LED with metasurface filter and spin-VCSEL with chiral reflector, the modulation of magnetization direction of spin injector 13 will induce an encoding not only on circular polarization (Pc) but also on light intensity, the combination of detection of circular polarization (Pc) and light intensity will provide a high single-to-noise ratio, which is especially important for high-speed data transmission.

The removing of optical component allows the fiber pigtailing with higher density of VCSEL devices. The light coupling in single mode fiber allows the polarization modulation technology can be compatible to the actual intensity modulation technology while benefiting the high polarization modulation speed in spin-VCSEL.

Those skilled in the art may understand that appropriate modifications can be made to various above-described circuit structures of the present disclosure as needed, all of which are within the scope of protection of the present disclosure.

Various embodiments of the present disclosure have been described above, and the foregoing descriptions are exemplary, not exhaustive, and not limiting of the disclosed embodiments. Numerous modifications and variations will be apparent to those of ordinary skill in the art without departing from the scope and spirit of the described embodiments. The terminology used herein was chosen to best explain the principles of the various embodiments, the practical application or improvement over the technology in the marketplace, or to enable others of ordinary skill in the art to understand the various embodiments disclosed herein.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A light emitting device comprising:
a semiconductor structure configured to generate light in response to carrier injection;
a spin injector configured to inject carriers into the semiconductor structure, wherein the light generated by the semiconductor structure has a circular polarization state determined by the magnetization state of the spin injector;
a magnetization controller configured to change the magnetization state of the spin injector; and
a chiral metasurface configured to make differential response to left circularly polarized light component and right circularly polarized light component of the light generated by the semiconductor structure.

2. The light emitting device according to claim 1, wherein
the semiconductor structure comprises gain medium of quantum dots or quantum wells configured to generate light upon receiving the carriers injected from the spin injector.

3. The light emitting device according to claim 1, wherein the spin injector is in a form of a bar-shaped channel, the magnetization controller comprises:
a first electrode and a second electrode respectively connected to two opposite ends of the bar-shaped channel to apply a current pulse into the bar-shaped channel, so as to change the magnetization direction of the spin injector,
wherein the spin polarization state of the carriers injected from the spin injector into the semiconductor structure is determined by the magnetization direction of the spin injector; and
the circular polarization state of the light generated by the semiconductor structure is determined by the spin polarization state of the injected carriers.

4. The light emitting device according to claim 3, wherein
the direction of the current pulse applied into the bar-shaped channel is capable of being reversely switched, and
the spin injector is configured such that its magnetization direction is capable of being switched by applying a current pulse with a direction opposite to that of the previous current pulse applied into the spin injector.

5. The light emitting device according to claim 1 further comprising a substrate, wherein
the semiconductor structure is formed above the substrate; and
the spin injector is formed above the semiconductor structure.

6. The light emitting device according to claim 5 further comprising:
a third electrode connected to the spin injector; and
a fourth electrode connected to the substrate,
wherein the third electrode and the fourth electrode are configured to apply a voltage between the spin injector and the substrate to inject carriers from the spin injector into the semiconductor structure.

7. The light emitting device according to claim 5, wherein
the chiral metasurface is arranged above spin injector.

8. The light emitting device according to claim 1, wherein
the chiral metasurface is arranged on the emission side of the light emitting device, and is configured to allow the light generated by the semiconductor structure to transmit through.

9. The light emitting device according to claim 1, wherein
the chiral metasurface is composed of chiral-shaped nanostructures.

10. The light emitting device according to claim 1, wherein
the chiral metasurface is configured to exhibit a higher transmittance for a first polarized light component compared to a second polarized light component, wherein the first polarized light component is either the left-handed circularly polarized light component or the right-handed circularly polarized light component, and the second polarized light component is the other opposite-handed circularly polarized light component.

11. The light emitting device according to claim 1 further comprising:
a bottom distributed Bragg reflector configured to reflect the light generated by the semiconductor structure, wherein the semiconductor structure is formed above the bottom distributed Bragg reflector, and the spin injector is formed above the semiconductor structure.

12. The light emitting device according to claim 11 further comprising:
a top distributed Bragg reflector formed above the spin injector and configured to reflect the light generated by the semiconductor structure, an intracavity resonant surface emitting laser structure is formed between the top distributed Bragg reflector and the bottom distributed Bragg reflector.

13. The light emitting device according to claim 12, wherein
a surface area of the spin injector is large enough to cover the semiconductor structure to ensure a homogenous carrier injection into the gain medium.

14. The light emitting device according to claim 12, wherein
the distance between the spin injector and the gain medium of quantum dots or quantum wells is configured to place the spin injector in one node of the stationary electromagnetic field formed by the light reflected from the top and bottom distributed Bragg reflectors.

15. The light emitting device according to claim 12, wherein
the chiral metasurface is formed above the top distributed Bragg reflector.

16. The light emitting device according to claim 15, wherein
the chiral metasurface is configured to exhibit a higher reflectivity for a first polarized light component compared to a second polarized light component, wherein the first polarized light component is either the left-handed circularly polarized light component or the right-handed circularly polarized light component, and the second polarized light component is the other opposite-handed circularly polarized light component.

17. The light emitting device according to claim 16, wherein the light emitting device is a spin-VCSEL with a chiral metasurface reflector, and has four lasing thresholds of carrier injection current satisfying the following relationship:
J_{T1}<J_{T3}<J_{T4}<J_{T2},
wherein the carrier injection current is the current of carriers injected upon the application of the voltage between the spin injector and the substrate,
J_{T1} is a first lasing threshold of carrier injection current for the first polarized light component when the spin injector is in a first magnetization state,
J_{T2} is a second lasing threshold of carrier injection current for the second polarized light component when the spin injector is the first magnetization state,
wherein, when the spin injector is in the first magnetization state, no light is emitted if the carrier injection current is lower than first lasing threshold J_{T1}, light having only the first polarized light component is emitted if the carrier injection current is between the first lasing threshold J_{T1} and the second lasing threshold J_{T2}, and light having both the first polarized light component and the second polarized light component is emitted if the carrier injection current is larger than the second lasing threshold J_{T2},
J_{T3} is a third lasing threshold of carrier injection current for the second polarized light component when the spin injector is in a second magnetization state, and
J_{T4} is a fourth lasing threshold of carrier injection current for the first polarized light component when the spin injector is in the second magnetization state,
wherein, the second magnetization state has an magnetization direction opposite to the first magnetization, when the spin injector is in the second magnetization state, no light is emitted if the carrier injection current is lower than the third lasing threshold J_{T3}, light having only the second polarized light component is emitted if the carrier injection current is between the third lasing threshold J_{T3} and the fourth lasing threshold J_{T4}, and light having both the first polarized light component and the second polarized light component is emitted if the carrier injection current is larger than the further lasing threshold J_{T4}.

18. The light emitting device according to claim 17, wherein
the voltage applied between the spin injector and the substrate is configured such that the carrier injection current J is between J_{T3} and J_{T4}:
$${J}_{T 1} < {J}_{T 3} < J < {J}_{T 4} < {J}_{T 2} .$$

19. The light emitting device according to claim 18, wherein
I₁>I₂,
I₁ is a first intensity of the light emitted from the light emitting device with the first polarized light component when the spin injector is in the first magnetization state, and
I₂ is a second intensity of the light emitted from the light emitting device with the second polarized light component when the spin injector is in the second magnetization state.

20. The light emitting device according to claim 19, wherein
the spin injector is switched into the first magnetization state when a larger intensity of light is expected to be emitted by the light emitting device; and
the spin injector is switched into the second magnetization state when a smaller intensity light is expected to be emitted by the light emitting device.
